(19) United States
(12) Patent Application Publication
(10) Pub. No.: US 2025/0218562 A1
(43) Pub. Date: Jul. 3, 2025

(54) MEDICAL ALERT SYSTEM BASED ON ENDOSCOPY ERGONOMIC RISK ASSESSMENT AND PREDICTION

(71) Applicant: ColoWrap, LLC., Durham, NC (US)

(72) Inventor: James HATHORN, Carrboro, NC (US)

(21) Appl. No.: 19/004,314

(22) Filed: Dec. 28, 2024

Related U.S. Application Data
(60) Provisional application No. 63/616,150, filed on Dec. 29, 2023.

(51) Int. Cl. G16H 20/30; G16H 10/60; G16H 20/10

(52) U.S. Cl. CPC G16H 20/30; G16H 10/60; G16H 20/10

(57) ABSTRACT

A medical alert system that provides alerts based on predictions of various potential risks to a patient, a physician, and/or medical staff for endoscopy procedures. The medical alert system includes a communication interface, memory, and at least one processor coupled to the memory and configured to receive, at the communication interface of the medical alert system, a request for a risk prediction for a future endoscopic procedure, where the request includes procedural information related to at least one of historical endoscopic procedure information or proposed endoscopic procedural information for the future endoscopic procedure. In response, the system outputs, via the communication interface, a risk prediction alert associated with performance of the future endoscopic procedure. The alert is based on predicted risks for one or more risks to a patient, a physician, and/or staff based on at least one of biographical information or the proposed endoscopic procedural information.

### CROSS REFERENCE TO RELATED APPLICATION(S)

This application claims the benefit of and priority to U.S. Provisional Application Ser. No. 63/616,150, entitled “Endoscopy Ergonomic Risk Assessment and Prediction” and filed on Dec. 29, 2023, which is expressly incorporated by reference herein in its entirety.

### INTRODUCTION

A colonoscopy is an example of an endoscopy procedure including an examination of the large intestine or colon through the use of a colonoscope. A colonoscope is a flexible, tube-like inspection device having a camera at its end. Colonoscopies are performed for a variety of medical reasons including detection of inflamed tissue, ulcers, abnormal growths or polyps, and colorectal cancer. Colonoscopy is increasingly used as a screening tool to detect colorectal cancer.

During a colonoscopy, as an example of an endoscopy procedure, a colonoscope is inserted into a patient's rectum and then advanced to the beginning of the colon (an area known as the cecum) in order to examine the lining of the large intestine. The efficiency and accuracy of this procedure is largely dependent on the case with which the colonoscope can be advanced. During the procedure, the colon may become over-distended or flopped in unnatural directions creating loops that hinder the advancement of the colonoscope and resulting in patient discomfort, longer examination times, and potentially inaccurate or incomplete screenings.

The difficulty in advancing the scope may be addressed by the application of manual pressure by a technician to manually support the patient's colon. The application of manual pressure is time-consuming and varies depending on the particular technician's strength, technique, endurance, and training. In order to apply differential pressure or to change the orientation of the colon within the body, the technician may roll the patient from the left side to a supine or to a prone position, which can be a difficult task with a sedated patient. The application of manual pressure and movement of the patient in order to support the patient's colon and advance the colonoscope during the procedure places a physical toll on the technician, which may lead to injuries to the technician. In addition, the application of manual pressure and movement of the patient may also cause injury to the patient.

### SUMMARY

In an aspect of the disclosure, a method and apparatus for predicting potential risks to the patient, physician, or staff in performing procedures used to examine the bowels including colonoscopy, sigmoidoscopy, and/or enteroscopy. Aspects presented herein provide a configuration for predicting a risk associated in performing endoscopic procedures using an artificial intelligence/machine learning (AI/ML) model.

The aspects presented herein may provide ergonomic interventions for at least one of the patient, physician, or staff to mitigate any potential risks to the patient, physician, or staff in performing the procedures. Aspects presented herein may assist in preventing injury to the patient, physician, or staff and reducing risks to the patient, physician, or staff during the procedures.

For example, a medical alert system provides alerts based on predictions of various potential risks to a patient, a physician, and/or medical staff for endoscopy procedures. The medical alert system includes a communication interface, memory, and at least one processor coupled to the memory and configured to receive, at the communication interface of the medical alert system, a request for a risk prediction for a future endoscopic procedure, where the request includes procedural information related to at least one of historical endoscopic procedure information or proposed endoscopic procedural information for the future endoscopic procedure. In response, the system outputs, via the communication interface, a risk prediction alert associated with performance of the future endoscopic procedure. The alert is based on predicted risks for one or more risks to a patient, a physician, and/or staff based on at least one of biographical information or the proposed endoscopic procedural information.

Additional advantages and novel features of aspects of the present invention will be set forth in part in the description that follows, and in part will become more apparent to those skilled in the art upon examination of the following or upon learning by practice thereof.

### BRIEF DESCRIPTION OF THE DRAWINGS

FIG. 1A is a schematic view of a colon with an endoscope (which may be referred to as a colonoscope for colonoscopy procedures) partially inserted therein.

FIG. 1B is a schematic view of a colon in which a sigmoid loop has developed due to an attempt to advance the endoscope against an unsupported colon wall.

FIG. 1C is a schematic view of a colon showing the application of manual pressure to the colon to facilitate insertion of an endoscope.

FIG. 2 is a diagram illustrating a system configured to receive input and provide a report with a prediction of risks in an endoscopic procedure, in accordance with aspects of the present disclosure.

FIG. 3 is a diagram of an example of an ergonomic risk analysis report, in accordance with aspects of the present disclosure.

FIG. 4 is a diagram of an example of an ergonomic risk analysis report, in accordance with aspects of the present disclosure.

FIG. 5 is a diagram of an example of an ergonomic risk analysis report, in accordance with aspects of the present disclosure.

FIG. 6 is a flowchart illustrating a method of a model to predicting risks in an endoscopic procedure, in accordance with aspects of the present disclosure.

FIG. 7 is a block diagram of a computer system configured to perform various aspects of the present disclosure.

FIG. 8 is a diagram illustrating a system configured to receive input and provide a report with a prediction of risks in an endoscopic procedure, in accordance with aspects of the present disclosure.

FIG. 9A and FIG. 9B illustrate example interfaces or devices that can be used to receive information from healthcare providers with feedback information relating to endoscopy procedures and the possible use of manual pressure, in accordance with aspects of the present disclosure.

FIG. 10A illustrates an example of the application of manual pressure to a patient during an endoscopy procedure.

FIG. 10B illustrates an example of a device for applying compression to the abdomen of a patient during an endoscopy procedure.

FIG. 11 illustrates an example of an application of a compression device to the abdomen of a patient for use with an endoscopy procedure.

FIG. 12 illustrates an example of a device for applying compression to the abdomen of a patient during an endoscopy procedure.

### DETAILED DESCRIPTION

For physicians that perform endoscopic procedures, more than 50% may report work-related injuries. A primary cause for the work-related injuries may be related to repetitive-motion associated with performing complex procedures. Looping, a primary barrier to performing safe and efficient colposcopies, is associated with use of peak push and torque forces on the scope and may cause musculoskeletal injuries, particularly to extremities used to advance the scope.

Numerous techniques, scheduling strategies, and technologies may be used in an effort to reduce injuries to endoscopy personnel and patients. As one, non-limiting example, a device may be applied to the abdomen of the patient to provide pressure or compression during the endoscopy procedure. In some aspects, the device may replace or supplement the use of manual pressure. Given the non-sterile nature of the endoscopy procedure setting, many of these technologies are single-use, and deployed prior to the procedure. To minimize unnecessary use of these technologies, it would be desirable to identify procedures likely to pose increased risks to physicians, staff, and/or patients in advance. As used herein, the term medical profession, healthcare professional, medical provide, or healthcare provider includes physicians, staff, and/or technicians that assist in medical procedures, such as endoscopy procedures.

Aspects presented herein provide an apparatus, method, and computer-readable storage medium for a medical alert system. For example, the medical alert system is configured to predict potential risks to the patient, physician, and/or staff in performing future procedures used to examine the bowels including colonoscopy, sigmoidoscopy, and/or enteroscopy. For example, an artificial intelligence (AI) or machine learning (ML) based medical alert system may be used to predict the potential risks to the patient, physician, and/or staff in performing such procedures. The AI/ML based medical alert system may utilize received input that relates to at least one of the patient, the physician, and/or staff to predict such potential risks. In response to detection of a potential risk that indicates a likelihood of an injury or complication for at least one of the patient or medical professionals, the system provides an alert for the potential risk. The medical alert system may also provide suggestions to reduce or minimize risk in connection with the procedure.

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details.

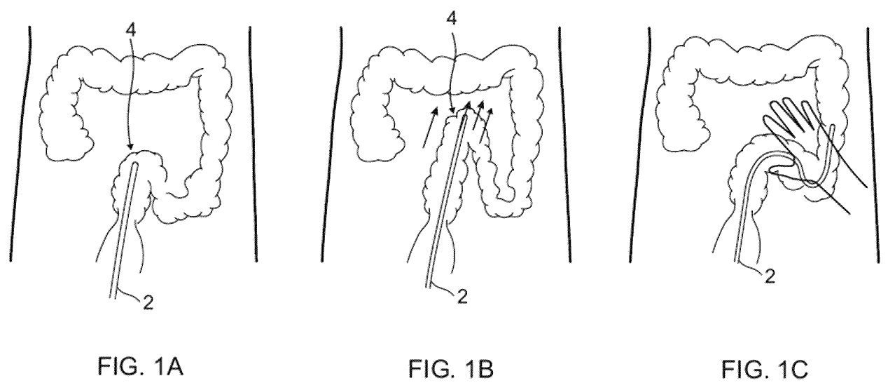

FIGS. 1A-1C, illustrate a sequence of steps of a colonoscopy, as one example of an endoscopy procedure. In FIG. 1A, a colonoscope 2 is inserted into the patient's rectum and advanced forward through the length of the colon. As the operator passes the colonoscope through the sigmoid region of the colon 4, the colonoscope may become impinged and cause distention and looping of the anatomy, as shown in FIG. 1B. The distention causes discomfort to the patient and increases the time required for the colonoscopy. In order to reduce the distended or looped area, a technician may apply manual pressure to abdomen of the patient. Among other examples, the technician may be a nurse, assistant, or other staff member. For example, the pressure may be applied by a nurse or surgical assistant as shown in FIG. IC.

The application of manual pressure is time-consuming and places a physical toll on the technician. The effectiveness of the manual pressure varies depending on the particular technician's strength, technique, endurance, and training. In order to apply differential pressure and change the orientation of the colon within the body, the technician may roll the patient from the left side to a supine or to a prone position, which can be a difficult task with a sedated patient. The application of manual pressure and movement of the patient in order to support the patient's colon and advance the colonoscope during the procedure may lead to injury of the patient or of the technician.

Many patients undergo colonoscopy while placed in the left lateral decubitus position on the stretcher or operating table. Additional information about the use of such manual pressure can be found in Prechel JA, Hucke R. Safe and effective abdominal pressure during colonoscopy: forearm versus open hand technique. Gastroenterol Nurs 2009; 32: 27-30; quiz 31-2, the entire contents of which are incorporated herein by reference. In applying manual pressure, the technician may reach over the patient from the opposite side of the table and to deploy pressure by placing their hands against the patient's sigmoid colon and then leaning backwards, using their bodyweight for leverage to exert force. While these methods are generally effective at generating pressure, they have also been identified as a causative factor for the high rate of work-related injuries among endoscopy nurses and staff. Physicians performing colonoscopy suffer work-related musculoskeletal injury at a particularly high-rate as well. The most frequent site of physician injury is the right upper extremity which experiences peak torque forces when while operators are attempting to advance the scope through (a looping) sigmoid colon. Additional details can be found in Spanarkel M, Hathorn J P. Looping During Colonoscopy: A Major, Implied Cause of Injury Among Endoscopy Healthcare Providers and a Proposed Solution, 2013, the entire contents of which are incorporated herein by reference.

In some instances, more than 50% of physicians that perform endoscopic procedures may report work-related injuries, with neck, shoulder, back, arm, wrist, and hand injuries being the most common. A primary cause for these work-related injuries may be based on repetitive-motion injuries associated with performing complex procedures using one-size-fits-all instruments. Looping, the primary barrier to safe and efficient colonoscopy, is associated with peak push and torque forces on the scope, and can lead to musculoskeletal injuries for the physician or other medical staff. For example, the push and torque force to guide the scope can cause strain to the right upper extremities (e.g., right shoulder, right arm, right elbow, right wrist, and/or right hand) as this is the arm that endoscopists often use to advance the scope. As used herein, an endoscopist may also be referred to as a physician, a doctor, a medical professional, and/or a healthcare professional.

Looping is most often addressed through staff-applied manual abdominal compression and patient repositioning. Staff-applied manual abdominal compression and patient repositioning can be physically demanding interventions, which may result in pain and injury to endoscopy staff that assist the endoscopists during the endoscopy procedure. In some instances, 85% of staff with the responsibility of staff-applied manual abdominal compression and patient repositioning have reported work-related injuries. Due to forces that the endoscopy staff apply in order to reposition the patient and/or apply manual pressure to the patient, these interventions may also pose a risk to patients, where complications may arise, such as but not limited to skin tears, bruising, hematomas, post-procedure pain, and in rare cases, life-threatening splenic rupture. In some instances, 25% of endoscopy staff have reported a patient complication due to manual pressure or patient repositioning.

Numerous techniques, scheduling strategies, and technologies may be used in an effort to reduce injuries to endoscopy personnel and patients due in part to significant burdens of ergonomic injuries to endoscopy personnel and risks to patients associated with manual interventions to mitigate looping. For example, one or more ergonomic interventions may be implemented by at least one of the patient, the physician, or the staff to reduce risks. Among other examples, the one or more ergonomic interventions may comprise at least one of the patient utilizing a compression device during the endoscopic procedure, receiving a sedation, or physical manipulation. In some aspects, the one or more ergonomic interventions may comprise at least one of the physician utilizing hand dials, an anti-fatigue mat, or increasing a scheduled time duration for the endoscopic procedure. In some aspects, the one or more ergonomic interventions may comprise at least one of the staff utilizing assistive equipment to assist in an application of external pressure on the patient or repositioning of the patient.

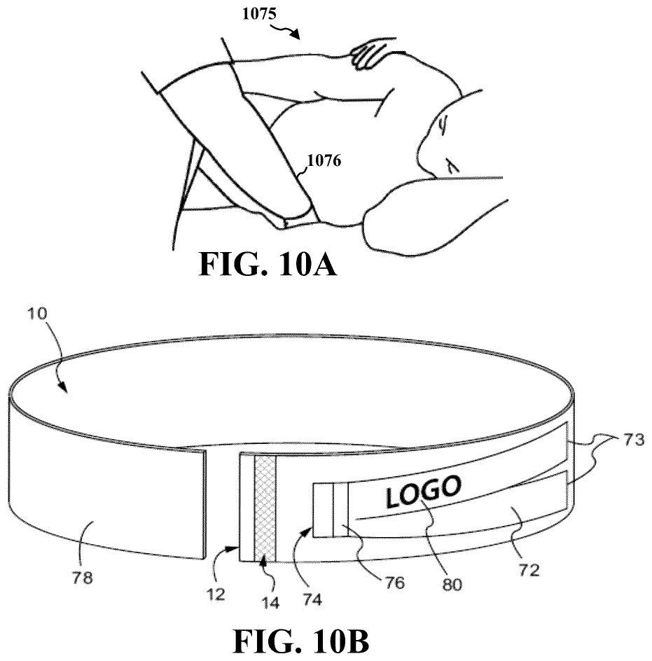

As one, non-limiting example of a strategy and technology to reduce injury, a compression device and/or an insert may be applied to the patient to apply general and/or targeted compression to the abdomen of the patient during an endoscopy procedure. The use of the device may take the place of or supplement the use of manual pressure, which may reduce the potential for injury to staff. As well, the uniform and/or targeted compression may assist in the endoscope movement and reduce the potential risk to the patient. FIG. 10A illustrates an example of a technician 1076 applying manual pressure to the abdomen of a patient, e.g. to assist with movement of the endoscope during an endoscopy procedure, such as illustrated in connection with FIG. 1C. As illustrated in FIG. 10A, the patient 1075 may be lying on their side, and may be sedated. The application of manual pressure is time-consuming and places a physical toll on the technician. The effectiveness of the manual pressure varies depending on the particular technician's strength, technique, endurance, and training. In order to apply differential pressure and change the orientation of the colon within the body, the technician may roll the patient from the left side to a supine or to a prone position, which can be a difficult task with a sedated patient.

In order to prevent looping and assist in insertion and/or withdrawal of an endoscope, and/or to improve imaging during an endoscopy procedure, a compression device may be placed on the patient. The device may include a primary elongated band or wrap of sufficient length for placement around a patient's lower abdomen. The primary wrap may include a closing mechanism, such as a hook and loop fastener material to secure the device around the patient and to apply an amount of broad support and compression. The device may also include one or more secondary straps that enable the technician to adjust the amount of compression applied by the device.

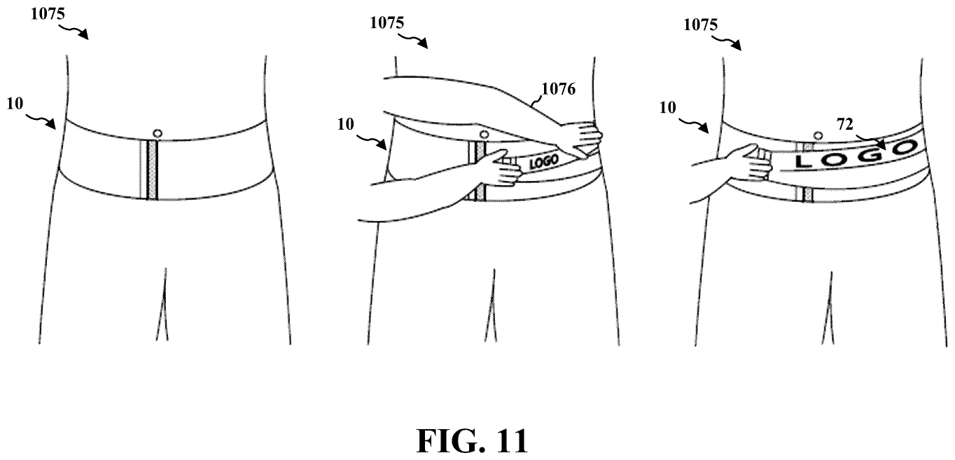

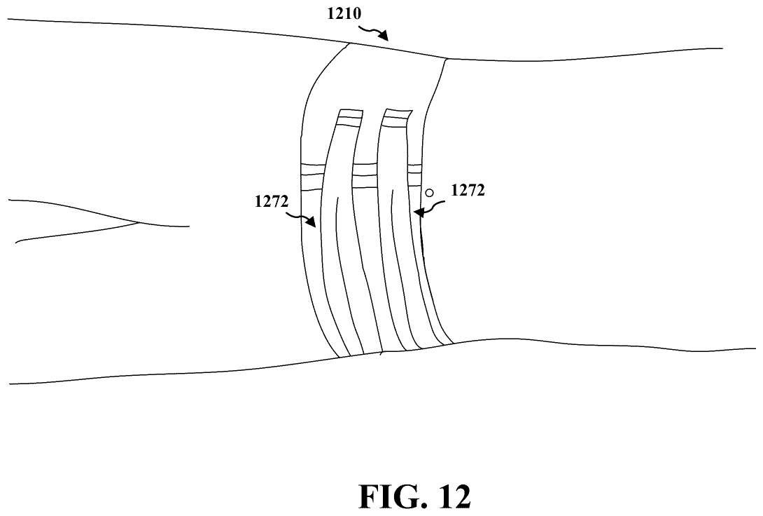

FIG. 10B illustrates an example device including a primary elongated band or wrap 10 of sufficient length for placement around a patient's lower abdomen. A closing mechanism 12 may be provided at the end of the primary band to secure the device around the patient so that it provides the desired amount of broad support and compression. A handle 14 may be sewn onto the exterior of one or both ends of the primary wrap to assist in fastening and closure. FIG. 11 illustrates an example application showing a primary elongated band or wrap 10 wrapped around an abdomen of a patient 1075. FIG. 10B and FIG. 11 also show a secondary strap 72 that can be pulled, or stretched, to apply added compression to at least a portion of the patient's abdomen. FIG. 12 illustrates another example compression device having a primary wrap 1210 and multiple secondary straps 1272.

For example, the primary wrap may be configured to have a width and length that allows it to be fastened around the patient's lower abdomen. The primary wrap 10 may comprise, entirely or in part, a flexible, bio-compatible foam, rubber, neoprene, polyester, nylon, non-woven or woven fabric, mesh fabric, synthetic fabric, microfiber fabric, silicon or vinyl plastic, or any other materials generally known to be used in medical fabrics and goods. The primary wrap 10 may be composed of both clastic and inelastic materials. In one example, the primary wrap 10 may comprise multiple layers laminated together. For example, the primary wrap 10 may comprise a neoprene layer and an outer fabric layer laminated on the neoprene layer. The fabric layer may enable the secondary strap to be removably fastened along the length of the primary wrap and/or strap 72 may also enable visual indicators 80 to be printed on the fabric, such as a nylon loop fabric. The primary wrap may also comprise an inner layer laminated on the neoprene. The primary wrap 10 may be placed around the patient's lower abdomen and secured using a closing mechanism 12 consisting of a strip of VELCRO® or hook material placed on the interior of the wrap 10 close to the location of the handle 14 on the opposite side. This hook strip may be fastened to the exterior side of the opposite end of the primary wrap 10.

The primary wrap may also accommodate an insert or attachment that provides specific support to one or more areas of the colon including the sigmoid, transverse, and cecal regions as well as the splenic and hepatic flexures. When the device is securely fastened, the secondary strap, the insert, or both, may be pushed, pulled, or otherwise pressed into the body in a manner that serves to support or ‘splint’ one or more areas of the colon including the sigmoid, transverse, and cecal regions as well as the splenic and hepatic flexures. Aspects described herein may be designed to provide broad lower abdominal support, and additional direct force to one or more areas of the colon including the sigmoid, transverse, and cecal regions as well as the splenic and hepatic flexures of a patient undergoing colonoscopy.

The addition of one or more appendages on the exterior of the primary wrap facilitates the application of additional directed force, e.g., without requiring adjustment of the primary wrap. The appendages may comprise one, or multiple, straps attached on one side to the edge or edges of non-clastic section. On the unattached end 74 of these straps 72 or 1272, there may be a handle, e.g., 76 shown in FIG. 10B. The handle or loop near the end of the strap may provide greater case to a technician, or added security, in pulling the elastic strap to apply targeted, additional compression. The strap(s) 72 may be pulled horizontally along the exterior of the primary wrap, and fastened using the closing mechanism securely enough to maintain tension. The one or more straps may be secured (e.g., sewn or otherwise attached) to the primary wrap as shown at 73 and may comprise an elastic material that is configured to be stretched and fastened to the primary wrap in order to apply targeted compression through elastic contraction of the material. The amount of targeted compression may be adjusted through adjustment of the position at which the removable end is coupled to the primary wrap, such as through a hook and loop type fastener. In some examples, the straps may be secured to an inelastic section of the primary wrap, and the tension generated by fastening these straps may cause additional compression of the inelastic section and/or an insert toward the body of the patient.

As depicted in FIG. 10B and 11, the secondary strap 72 may allow nurses and technicians to easily adjust and readjust the force on a particular region of the patient's abdomen, such as the sigmoid colon and/or the transverse colon among other example regions, from the location in the procedure or operating room that these staff members typically occupy, relative to how patients are often positioned during an endoscopy procedure. The endoscopy compression device described herein additionally eliminates the need for the nurse or assistant to provide manual abdominal compression, thereby reducing their risk of musculoskeletal injury. Additional, manual compression may be applied along with compression from the device. The device is designed to be quickly and easily removed should the need arise.

In order to apply pressure, e.g., compression, to the abdomen, one or more inserts may be placed between at least a portion of the device and the patient's abdomen. The insert may be configured to communicate compression from the primary band and/or secondary strap to the abdomen of the patient. The insert can be used in combination with the compression device to improve the efficacy of the compression. The insert(s) may assist in applying a more consistent amount of support to patients of varying body mass indexes. In some aspects, the insert may assist in maintaining a position of the sigmoid and/or transverse regions of the colon during an endoscopy procedure. In some aspects, the insert may include a density similar to adipose tissue to help communicate compression provided by a band around the lower abdomen to the colon in order to prevent looping and assist in insertion, withdrawal and/or visualization during an endoscopy procedure.

Given the non-sterile nature of the endoscopy procedure setting, many of these technologies, such as a compression device and/or insert may be single use devices that are deployed prior to the procedure. To achieve a more efficient use of these technologies, it is desirable to identify procedures likely to pose increased risks to physicians, staff, and/or patients in advance.

Aspects presented herein provide an apparatus configured to predict various potential risks to the patient, physician, and/or staff in performing procedures used to examine the bowels of the patient, e.g., including colonoscopy, sigmoidoscopy, and/or enteroscopy. For example, an AI/ML based medical alert system may predict the potential risks to the patient, physician, or staff in performing such procedures and provide an alert that assists the physician or staff in selecting and applying assisting technologies such as compression devices and/or inserts, and/or in making physical adjustments that are predicted to avoid or reduce potential injuries or complications. The AI/ML system may utilize received input that relates to at least one of the patient, the physician, or the staff to predict such potential risks and trigger the alert. These AI/ML tools may be configured to process the input received related to at least one of the patient, physician, and/or staff to determine such potential risks. At least one advantage is that the apparatus may provide ergonomic interventions for at least one of the patients, the physician, and/or the staff to mitigate/reduce various potential risks to the patient, the physician, and/or the staff in performing the procedures. Aspects presented herein may further provide alert for the ergonomic interventions or predicted risks in advance of the endoscopic procedure based at least on information related to the patient, the physician, and/or the staff. In some aspects, the predictions and alert may be based on a combination of information about the particular physician, information about the particular patient, and information about the particular staff to predict a potential risk that is specific to the particular procedure for the particular patient.

Aspects presented herein provide a medical alert apparatus or system configured to provide a colonoscopy physical intervention assessment, analysis, and/or risk-based algorithm generation tool. In some instances, the apparatus is configured to receive data, perform an analysis, and generate a clinical algorithm and/or identify future procedures likely to pose increased risks to patients and endoscopy staff due to the need for manual, physical intervention, such as but not limited to manual abdominal compression and patient repositioning. The apparatus may allow users to set and adjust a risk-tolerance level which will broaden/narrow those procedures identified as “high-risk”. For example, the system may include an input component or communication interface that is configured to receive an indication of the risk-tolerance level or setting from the user and to adjust the prediction algorithm accordingly. At least one advantage of the disclosure is that the apparatus may allow endoscopy units to be aware of endoscopy procedures that are predicted to likely demand additional resources and/or additional protective equipment or devices to protect medical staff and patients.

There may be primary patient factors that are predictive of a more difficult colonoscopy. A difficult colonoscopy may be defined by cecal intubation time as prolonged cecal intubation time (e.g. defined by a duration threshold related to the amount of time during the endoscopy for the endoscope to reach the location of the patient's cecum). The medical alert system presented herein may make predictions to identify which colonoscopies are more likely to require physical staff intervention (e.g., manual abdominal pressure and patient repositioning) to address looping or to assist the endoscope movement to the cecum, and yet may be focused on patient factors only. However, such considerations may not include both a prediction of the likelihood of the need for manual pressure and repositioning in specific colonoscopies and an assessment of the risks to endoscopy staff and/or patients associated with these interventions.

Ergonomic risks may be a function of force, duration/frequency, or posture. For example, staff ergonomic risk drivers may be associated with manual pressure and intensity. The physical characteristics of the staff may be considered, such as the physical size, strength, and/or any prior injuries or physical limitations of the medical professionals involved in the endoscopy procedure (e.g., the physician or endoscopist and/or any additional medical professionals or staff). For example, the role of the staff member during the procedure may affect the risk of injury to the staff and/or patient in performing the procedure. The duration of applying manual pressure may be a function of physician experience and preference, in some aspects. The physician's experience and habit may contribute in determining how/when these interventions are applied. The duration of applying manual pressure may also be a function of the procedure difficulty (e.g., looping). Manual pressure and repositioning combat looping, which is the primary cause of difficult, prolonged cecal intubations during colonoscopy. The amount of manual force that is applied may be a function of patient body habitus and/or weight. For example, the higher the patient weight, the greater the force applied by staff to prevent looping or assist in the endoscope movement. The posture of the staff while applying the manual pressure may be a function of the room set-up, assistive equipment, or an interaction of physician stature and staff stature. For example, the room set-up (e.g., floor space, physician display location, etc.) may or may not provide sufficient space for staff to maneuver around the patient, physician, and/or equipment to apply the manual pressure. Assistive equipment may comprise a determination as to whether stools available for staff or the physician, or whether the patient bed height adjustable. The interaction of physician stature and staff stature may comprise a determination of whether the height of the physician and/or the staff is complementary. For example, a tall physician may adjust the height of the bed to suit the height of the doctor, such that a short staff member assisting the physician may have difficulty in applying manual pressure due in part to the height of the bed accommodating the tall physician and not the short staff member.

In some instances, ergonomic risks may be associated with patient repositioning. For example, staff ergonomic risk drivers may be associated with the manual repositioning of the patient. The physical characteristics of the staff may be considered, such as the physical size, strength, and/or any prior injuries or physical limitations. In addition, the role of the staff member during the procedure may affect the risk associated to the staff in performing the procedure. The frequency of staff manually repositioning the patient may be a function of physician experience and preference. The physician's experience and preferences may contribute in determining how/when the staff is to reposition the patient during the procedure. The frequency of repositioning the patient during the procedure may also be a function of the procedure difficulty (e.g., looping).

Multiple factors may be utilized in determining the difficulty of the procedure for either the frequency of repositioning the patient during the procedure or the duration of applying manual pressure such as, but not limited to, patient factors, physician experience, physician technique, equipment, and sedation method. Some patient factors that may contribute to determining the difficulty of the procedure may include age, gender, race, height, weight, body mass index, waist circumference, waist-to-height ratio, functional bowel disorders, diverticulosis, surgical history (e.g., abdominal region), medications (e.g., current, past), abdominal anatomy, skeletal structure, or the like. These non-limiting factors may be found in the patient's records. However, such information may be supplemented by the patient self-reporting the data in advance of the procedure. This self-reported patient data may be captured via a phone screening call where a nurse or staff speaks with the patient to confirm information prior to the procedure or via an app or web interface. Applications may be particularly popular for colonoscopy, as achieving effective bowel preparation is important and may depend on the patient following instructions. This type of application may also be used to capture patient self-reported data relevant to identifying patients likely to have a difficult colonoscopy. In some instances, the application may allow the patient to provide photos of their abdomen in order to assess abdominal, visceral, and/or subcutaneous tissue volume and general abdominal anatomy of the patient as input to the risk assessment model. In some instances, the area and volume of some tissue may factor into the determination of the difficulty of the planned (e.g., future) endoscopy procedure. For example, the area and volume of the total abdominal tissue, visceral adipose tissue, or the subcutaneous adipose tissue may have a correlation to the difficulty of the endoscopy procedure. These measurements may provide an accurate prediction as to whether a colonoscopy is going to be a difficult colonoscopy. In some instances, these measurements may be estimated based on photographs of the patient's abdomen from one or more different profiles that could be captured by patients and submitted along with other self-reported measures in advance of their colonoscopy, all of which could be submitted into the risk analysis system.

The amount of manual force exerted by the staff to reposition the patient during the procedure may be a function of at least one of the patient body habitus/weight, sedation method, or assistive equipment/personnel. For example, the higher the patient weight, the greater the force exerted by staff to reposition the patient during the procedure. In some instances, patients under deep sedation do not provide any assistance of shifting or moving their body during the procedure, such that staff must reposition the patient during the procedure. In some instances, an example of assistive equipment or personnel may include a slide-sheet configuration where a slide-sheet is placed under a patient (e.g., under deep sedation, elevated weight, etc.) to allow staff to utilize the slide-sheet to reposition the patient during the procedure, but this technique is a two-person operation and would require additional staff be present during the procedure. The posture of the staff while repositioning the patient during the procedure may be a function of the room set-up or an interaction of physician stature and staff stature. For example, the room set-up (e.g., floor space, physician display location, etc.) may or may not provide sufficient space for staff to maneuver around the patient, physician, and/or equipment to reposition the patient. The interaction of physician stature and staff stature may comprise a analysis, calculation, or determination of whether the height of the physician and/or the staff is complementary. For example, a tall physician may adjust the height of the bed to suit the height of the doctor, such that a short staff member assisting the physician may have difficulty in repositioning the patient during the procedure due in part to the height of the bed accommodating the tall physician and not the short staff member.

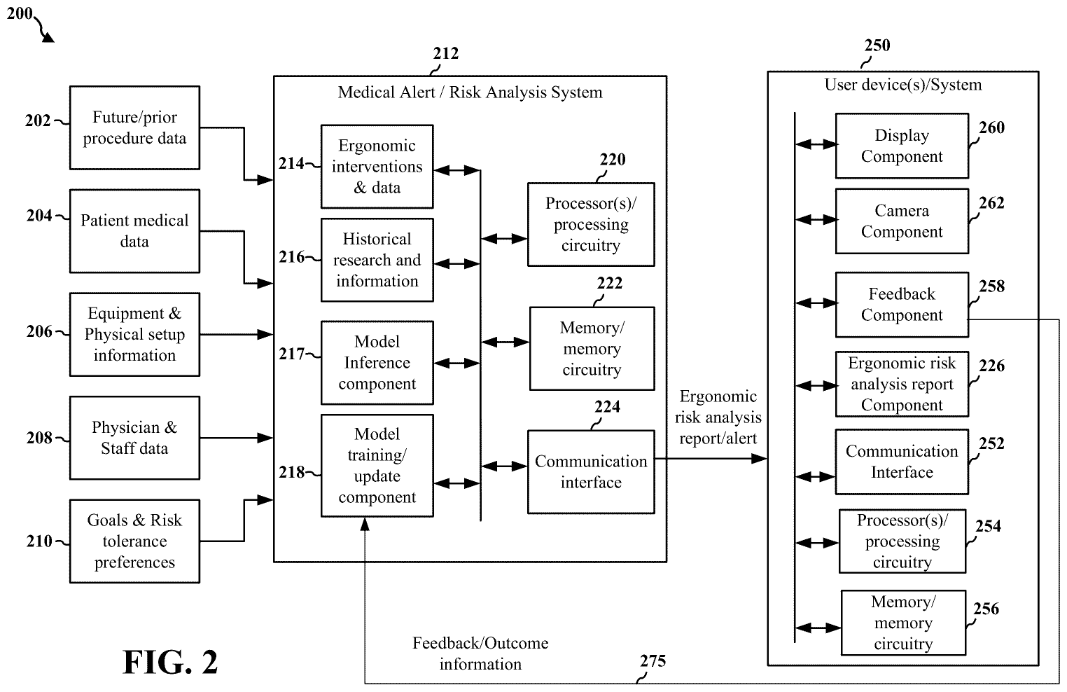

FIG. 2 illustrates a diagram 200 showing an example of a risk analysis system 212 that is configured to predict or identify one or more risks in an endoscopic procedure. In some aspects, the risk analysis system may make the predictions based on the collection and storage of historical information. In some aspects, the predictions may be based on a model using historical information, such as an AI/ML model. In some aspects, the risk analysis system 212 may be comprise an AI and/or ML (AI/ML) model that receives input and provides a risk prediction inference based on a trained AI/ML model. The risk analysis system 212 may also be referred to as a medical alert system, for example. In some aspects, the risk analysis system 212 may include a convolutional neural network (CNN) that receives information so as to continuously or periodically train an AI/ML model used by the CNN to predict or identify the one or more risks to at least one of the patient, physician, or staff involved in the endoscopic procedure. The risk analysis system 212 may comprise one or more processors 220 (or processing circuitry), memory 222 (or memory circuitry), where the one or more processors 220 are configured to cause a computer system to perform the aspects described herein in connection with the use of the AI/ML model, as described herein. The risk analysis system 212 may further include a communication interface 224 that is configured to receive input (e.g., 202, 204, 206, 208, 210) and provide output from the risk analysis system 212. The output may be a prediction determined by the model inference component 217 after the AI/ML model is trained by the model training/update component 218. The communication interface 224 may include a network interface, a communications port, and/or other components to enable the exchange of communication via a communication path (e.g., whether wire, cable, fiber optic, wireless link, and/or other communication channel between computer systems), which enables the risk analysis system 212 to receive input from one or more devices and to provide output to one or more devices. In some aspects, the risk analysis system may be configured to receive input from multiple remote user devices and to provide output to one or more remote user devices. In some aspects, the output may be provided to one or more of the user devices that provided input to the risk analysis system 212. The risk analysis system 212 may include one or more AI/ML models. In some aspects, the risk analysis system 212 may have a model configured to identify risks to at least one of the patient, physician, or staff associated with performing endoscopy procedures. For example, for a particular planned endoscopy procedure, the risk analysis system 212 may be configured to identify risks and/or provide risk reducing recommendations for a particular endoscopy procedure (e.g., a procedure for a particular patient). For example, the risk analysis system 212 may be configured to provide and alert that identifies risks to at least one of the patient, the physician, or the staff and/or provide ergonomic interventions that are targeted to a particular patient or a particular procedure in an effort to mitigate or reduce the identified or predicted risks.

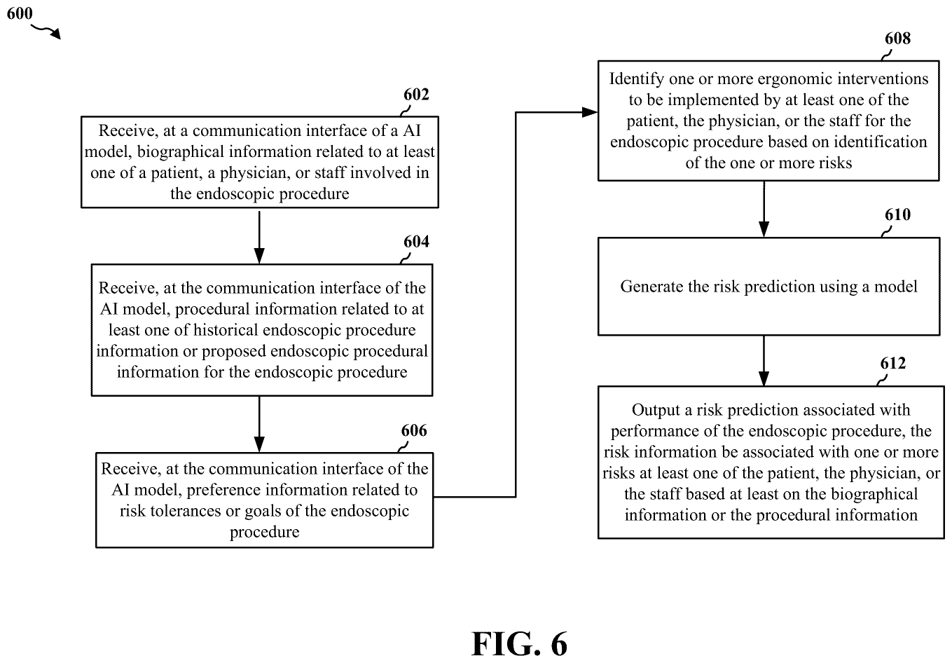

In some instances, the system 212 may receive a request to identify potential risks to at least one of the patient, physician, or staff based on one or more inputs. For example, an input may comprise future and/or prior procedure data 202. As an example, the request may correspond to 602 and/or 604 in FIG. 6. As an example of prior procedure data for a particular patient, the input at 202 may include information related to the procedures the patient may have had conducted in the past and/or procedures the patient may be scheduled for in the future. The historical data of the patient may be processed to indicate any risks that were involved in the performance of such procedures, such that the system 212 may include such historical information in predicting or identifying risks. As an example, the prior procedure data for a medical professionals, e.g., which may include a combination of a physical and one or more additional medical professionals, a facility layout, equipment, etc. may be considered to predict potential risk or recommend interventions (such as the use of a compression device and/or insert) in connection with future endoscopic procedures by the same group of medical professionals at a same facility to reduce the potential for complications or injury to the patient, physician, and/or medical staff. In some aspects, the future data may also include information related to procedures that may be scheduled for the patient. The future data may indicate information related to risks that may be present in performing the future procedure. In some instances, the patient medical data 204 may include medical history of the patient, such as but not limited to height, weight, medical/physical history, or the like which may be utilized by the system 212 to identify or predict various risks based on historical data for other patients and their corresponding medical history. In some instances, equipment and physical setup information 206 may be provided to the system 212. For example, the equipment and physical setup information 206 may include information related to the physical layout of the location (e.g., treatment room) where the endoscopic procedure will take place. In another example, equipment and physical setup information 206 may include information related to the equipment that may be available and/or utilized during the procedure. The information related to the equipment and physical setup information 206 may be utilized by the system 212 in identifying/predicting various potential risks. In some instances, an identification of the physician and staff data 208 may be provided to the system 212. For example, the physician and staff data 208 may include information related to the physician and staff that are scheduled to perform the procedure. In some instances, the physician and staff data 208 may include historical information related to the number of times the physician and/or staff have performed procedures. The physician and staff data 208 may include future information related to the procedures that the physician and/or staff may be scheduled to perform in the future. In some instances, the physician and staff data 208 may include information related to ergonomic interventions that may have been used by any of the physician and/or staff in previous procedures, or physical capabilities or limitations of the physician and/or staff. For example, the historical procedure information that is used to train the model (or feedback information that is used to refine the model) may include whether an intervention was used and the associated outcome. Among other examples of potential interventions, such potential interventions may include the use of a compression device (such as described in connection with any of FIGS. 10B-12 and/or an insert). The physician and staff data 208 may be utilized by the system 212 in identifying/predicting various potential risks. In some instances, the goals and risk tolerance preferences 210 may be provided to the system 212. For example, the goals and risk tolerance preferences 210 may include information related to the goals of the procedure and/or risks that may be tolerated during the procedure. The goals and risk tolerance preferences 210 may be utilized by the system 212 in identifying any risks.

The information may identify information about the procedure, the physical location or site of the procedure, the physician, the staff, the outcome, and/or any complications or challenges associated with the procedure. As another example of prior procedure data that may be input at 202, the historical data may include data for prior procedures performed for other patients, which may be used to train and/or update the AI/ML model. For example, the input may include information for the same location, the same physician, the same staff, and/or the same procedure, among other examples. In some examples, the input may include information for other locations/sites, other physicians, other staff, and/or other procedures. The model training/update component 218 may use such input to train the AI/ML model in order to improve the accuracy of model inferences for a particular patient. In some aspects, the information used to train the AI/ML model may avoid input of any information that would personally identify a particular patient. For example, the patient information may include height, weight, BMI, waist circumference, waist-to-height ratio, functional bowel disorder information, diverticulosis information, medication information, abdominal anatomy or skeletal structure information, total abdominal tissue area and/or volume information, visceral adipose tissue area and/or volume information, and/or subcutaneous adipose tissue area and/or volume information without a name, patient number or other information that would personally identify a particular patient. The information for other prior procedures may include other information described in connection with 204, 206, 208, and/or 210 for the other procedures. The prior procedure data may include outcome information for the prior procedures, e.g., including an identification of challenges, complications, or injuries to patient, physician, or staff. The prior data may be used to train the AI/ML model, e.g., to predict or infer potential risks for a particular future procedure based on an analysis of the historical procedure information (e.g., prior data). As illustrated at 216, the risk analysis system 212 may include a component that receives and stores historical information (e.g., which may include historical research and other historical information). The historical information may be used to train and/or update the AI/ML model.

The system 212 may process (e.g., pre-process) the input (e.g., 202, 204, 206, 208, 210) and provide the processed input to the model training/update component 218. The system 212 may be trained based on historical training information provided to the model training/update component 218 (e.g., which may include information specific to a physician, specific to a facility, specific to a medical group (which may allow for a targeted prediction of risk) and/or information from a broad group of various physicians, various facilities, and/or various medical groups (which may allow for a more comprehensive risk prediction and/or recommendations for interventions that have overcome, avoided, or reduced the predicted risk). The system 212 may be trained to identify or predict risks based on the input to the system 212. Once trained using the historical information, the system 212 may receive the input for a particular procedure for a particular patient and may provide risk prediction output for the particular procedure. For example, the system 212 may identify a risk based on an elevated body mass index of the patient that may be included within the patient medical data 204, such that the system 212 may recommend an ergonomic intervention. For example, the system 212 may process the input and output a recommended ergonomic intervention based on the ergonomic interventions and data 214, in an effort to mitigate or reduce the risk in performing the procedure on a patient having an elevated body mass index. In another example, the system 212 may identify a risk in instances where the staff or physician scheduled to perform the procedure may experience difficulty in physically repositioning the patient during the procedure due to the staff and/or physician being much smaller and/or weaker than the patient and unable to physically reposition the patient during the procedure. In such instances, the system may suggest an ergonomic intervention, such as the patient utilizing a compression device that minimizes or prevents looping, or lessens the physical exertion of the physician and/or staff to reposition the patient during the procedure.

The system 212 may output an ergonomic risk analysis report (which may also be referred to as an alert) via the communication interface 224 which may indicate the identified risks, as well as the recommended ergonomic interventions that may accommodate for the identified risks. In some aspects, the report may be output to a user device 250 or user system, which may also include a communication interface 252, memory 256, processor circuitry 254, and an ergonomic risk analysis report component 226 configured to receive the report. In some aspects, the user device 250 may further include a display 260, and may be configured to display a visual representation of the ergonomic risk analysis report to a user at the display 260.

In some aspects, the user device 250 may further include a feedback component 258 that is configured to provide feedback information 275 regarding the outcome of the particular procedure, which may include the use of the recommended interventions. The feedback may be provided to the model training/update component 218 to enable further training or update of the AI/ML model. This enables the model to be updated, trained, or refined in an ongoing manner in order to improve the accuracy of the predictions and recommendations for future endoscopic procedures.

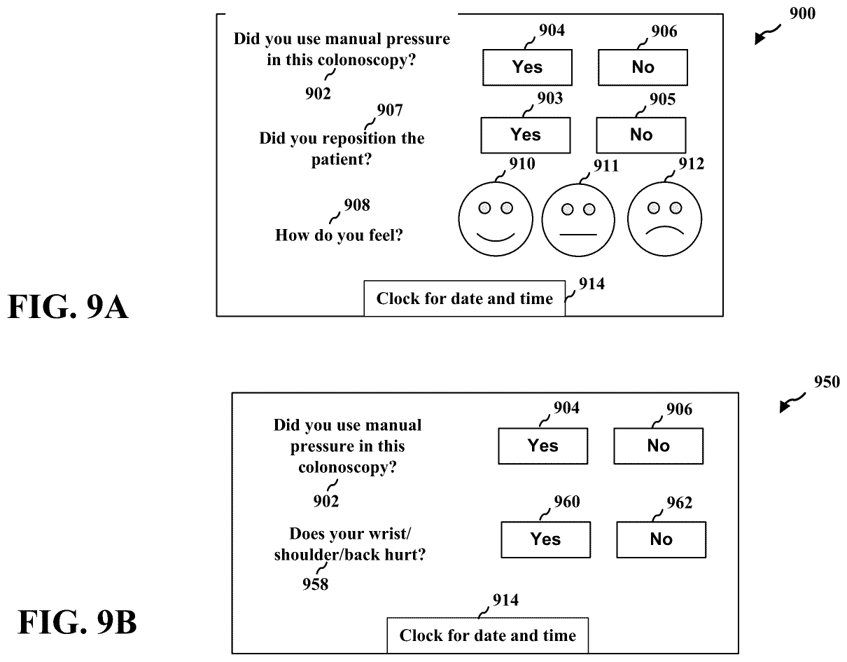

In some aspects, feedback relating to an endoscopy procedure may be received via one or more devices located at or near the procedure site. For example, FIG. 9A and FIG. 9B illustrate example interfaces or devices that can be used to receive information from healthcare providers with feedback information 275 relating to endoscopy procedures and the possible use of manual pressure. As an example, a computer interface, touch screen, or electronic device with manual selection buttons may be conveniently located inside of a treatment room, just outside a treatment room, or at a nearby desk, station, or kiosk, to enable a healthcare professional involved in an endoscopy procedure to quickly input information that may not be captured in an electronic medical record for the patient's procedure. The device may be configured with a simple user interface 900 that allows the healthcare professional to easily provide the information in a simple and intuitive manner. FIG. 9A shows, at 902, that the device may display (whether electronically or in physical print) the question that requests input about whether manual pressure was used for the endoscopy procedure. Multiple selection options may be presented to the user. In an example, two buttons may be provided, e.g., one for a positive response, at 904, to the question and another for negative response, at 906. If the device includes a user interface, the buttons 904 and 906 may be selected via user touch at a touchscreen. In other examples, the buttons 904 and 906 may be manual buttons that the user presses to make their selection. The device may include memory and/or a processor that stores the user selection. As well, as shown at 914, the device may include a clock that maintains a time and date. This enables the information to be stored with a timestamp and associated with a particular endoscopy procedure based on the timestamp. For example, the feedback information obtained via the device may be associated with a particular endoscopy procedure based on the location of the device (e.g., within a treatment room or just outside of the treatment room) and the timestamp information without requiring the healthcare professional to input further identifying information. The user interface 900 may ask additional questions to solicit further information about the effects of manual pressure on the healthcare provider. FIG. 9A illustrates an example question 908 that asks “How do you feel?” and includes simple selection options, such as a smile 910, a neutral face 911, or a frown 912. Similar to the buttons 902 and 904, the options may be provided for user selection at a touch screen or manual buttons that are selected by pressing the button. The selections may also have visual indicators or differentiators, such as color. For example, the smiling face option (e.g., 910) may be green, the neutral face (e.g., 911) may be yellow, and the frowning face (e.g., 912) may be red, as a non-limiting example. The user selection may light up once selected to assist the user in making a correct selection. The device may present various questions to the healthcare professional, such as presenting or displaying a question about whether the patient was repositioned, as shown at 907, with user selection buttons 903 and 905 to receive an answer from the user.

FIG. 9B illustrates additional example aspects of an interface 950 for a device similar to FIG. 9A to receive user feedback following an endoscopy procedure. Similar components are illustrated with the same reference number in FIG. 9A and 9B, and a device may include any combination of aspects from FIG. 9A and FIG. 9B. FIG. 9B illustrates that the presented questions may ask for more targeted feedback about the effect of manual pressure or repositioning on the medical professional. For example, the device may display a question asking “Does your wrist/shoulder/back hurt?” as shown at 958 and provide user selectable buttons 960 and 962 to receive the user response. Although 958 shows the inquiry as a single, combined question, each inquiry may be displayed separately to allow for separate user responses. For example, the device may display the question “Does your wrist hurt?,” “Does your shoulder hurt?,” and “Does your back hurt?” with separate response buttons for each inquiry. In some aspects, the device may be connected via a communication interface (e.g., such as the internet or other network) and configured to log the input with the time stamp. In other aspects, the device may log, or store, the input locally with the time stamp. The data can then be accessed and cross-referenced to match, or correlate, the input feedback to a specific endoscopy procedure and the other procedure related data generated by an export from a patient's electronic medical record (EMR.) This would allow for consideration of relationships between patient and procedure characteristics and outcomes like staff-reported musculoskeletal pain in the refinement of the mode, e.g., at 218.

In some aspects, additional procedure information, or feedback, may be obtained via a sensor or camera without manual input by a user. For example, a camera may be positioned in the treatment room, and the user device 250 (or user system) may include a camera component 262 that receives the video from the camera. The video of the endoscopy procedure may be used to identify and annotate certain behaviors. In some aspects, the analysis of the video may be performed by the AI/ML model to identify and annotate certain behaviors, postures, or positioning, etc. that can then be correlated with the procedure records from the EMR. In some aspects, the analysis of the video may be analyzed in real-time, and the system may provide an alert in real-time to the medical professional, such as through a sound, a light, or a vibration to warn them to change their posture or position. For example, the alert may be provided as a vibration in a watch worn by the medical professional, a light in the treatment room, or a sound.

In some aspects, the risk analysis system 212 may be provided at a central server that is accessed by one or more remote terminals to receive input for model training and/or model inference. The risk prediction report may be output via a communication interface from the server to one or more remote user terminals (e.g., 250). In some aspects, the AI/ML inference may be performed at a remote server and provided to the user device. As an example, the prediction report may be accessed via a website or an application. In some aspects, the AI/ML model component may be local at a user's device or an application within a local network or user system. In some aspects, the AI/ML model component may be accessed as an add on program (or add in tool) that adds additional functionality to a user system, such as a medical software system. In some aspects, the output may indicate a flag or alert that is provided at patient check in, enabling one or more interventions to be prepared for a procedure. In some aspects, the flag or alert may be provided in advance of a procedure, e.g., in advance of check in.

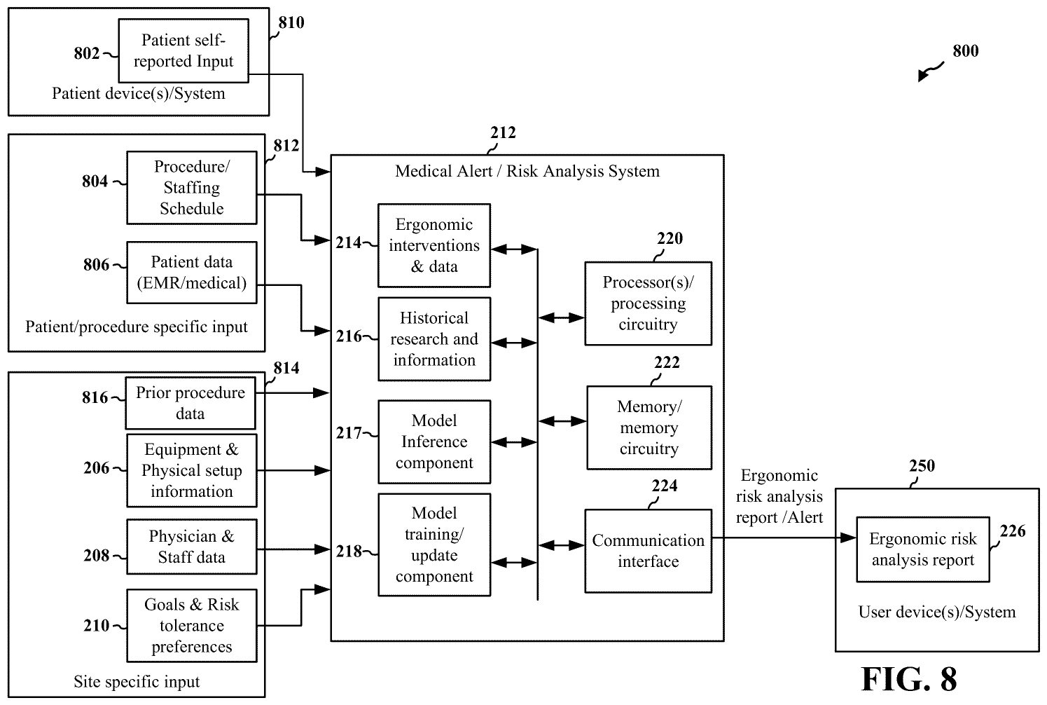

FIG. 8 illustrates an example 800 of the risk analysis system 212 of FIG. 2 illustrating the different types of input that may be received. In some aspects, the input may include patient self-reported input 802 that may be received at the system 212 from a remote patient device 810. For example, the patient may input some information via a website or an application. As an example, the patient may input a picture or photo, such as a photo of an abdominal region of the patient.

At 812, information may be input that is specific to the patient and/or procedure for which the prediction is requested. For example, the information for the particular patient may include patient data 806 or electronic medical record (EMR), e.g., medical information. The procedure and/or staffing information for the particular procedure may be input at 804. The information input at 812 may be received from a different device than the information at 802.

As illustrated at 814, site specific information may be provided, e.g., including prior procedure information 816, equipment and physical set up for the particular site, historical physician and/or staff data for the site, goals and risk tolerance preferences for the site and/or customer requesting the prediction. In some aspects, the site specific input may also include prior outcome or challenge information associated with prior procedures. Other aspects of FIG. 8 that may be similar to the system described in connection with FIG. 2 are illustrated with a same reference number.

For example, the ergonomic risk analysis report may include a scoring of risks for at least one of the patient, physician, or staff in performing the procedure. For example, the risk analysis report may indicate a risk rating (e.g., low, medium, high) for at least one of the patient, physician, or staff based on the procedure to be performed (e.g., colonoscopy) and/or their specific role during the procedure. In some instances, the risk analysis report may indicate a risk rating (e.g., low, medium, high) for at least one of the patient, physician, or staff based on the role during the procedure over a previous period of time. In some instances, the risk analysis report may indicate an individualized risk rating (e.g., low, medium, high) for at least one of the patient, physician, or staff over a previous period of time. The report, and/or the visual representation of the report, may include various examples of visual representations to assist in the mitigation of risks associated with endoscopy procedures.

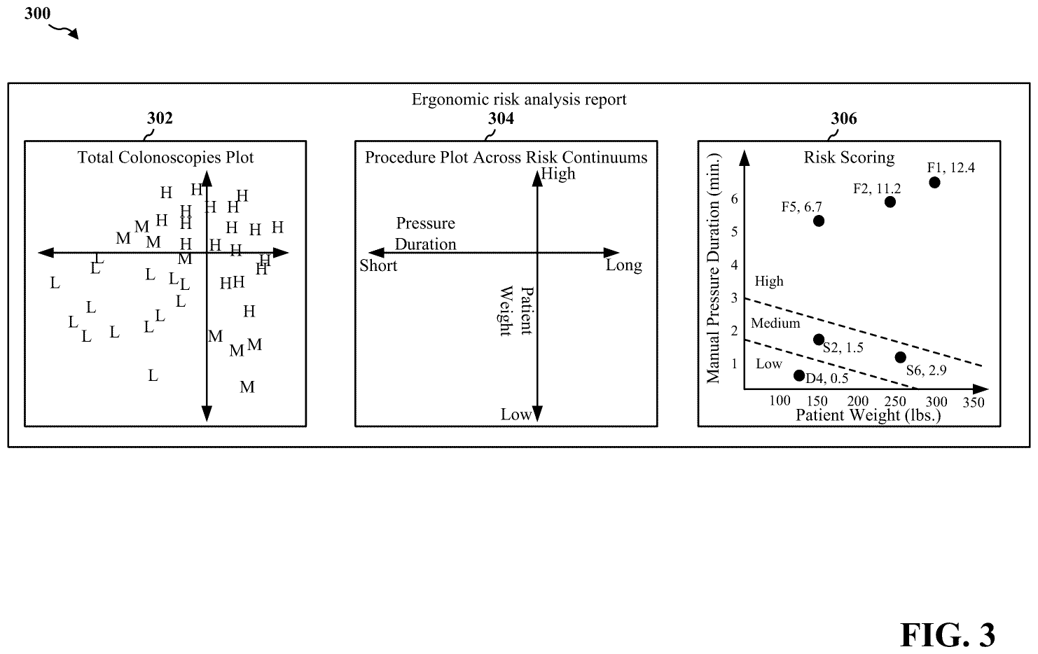

FIG. 3 illustrates a diagram 300 showing an example of visual representations that may be provided with an ergonomic risk analysis report. The example ergonomic risk analysis report of FIG. 3 may include a total procedures plot 302 comprising a plot of total procedures (e.g., colonoscopies) and a plot 304 comprising a procedure plot across risk continuums. In the plot 302, an amount of total colonoscopies may be plotted showing their respective level of risks. For example, all the high risk, medium risk, and low risk colonoscopies may be plotted in plot 302. In some instances, the plot 302 may indicate the risk based on manual pressure and/or repositioning. In some instances, the plot 302 may indicate the risk based on other factors discussed herein. The plot 304 may indicate the risk of manual pressure and/or repositioning as a function of time duration and patient weight. For example, a short pressure duration and a low weight patient may be considered as a low risk, whereas a long pressure duration and a high weight patient may be considered as a high risk. In some instances, a pressure duration that may be in between the short duration and long duration, while the patient has a body weight that is between the low weight and high weight may be considered as a medium risk.

In some instances, the ergonomic risk analysis report may comprise a scoring for the physician or staff involved or scheduled for a procedure. For example, the ergonomic risk analysis report may comprise a plot 306 that comprises a risk score for personnel involved in the procedure. For example, the plot 306 may indicate a risk scoring based on the patient weight and a time duration of manual pressure. The plot 306 may indicate a risk scoring for specific personnel based on the weight of the patient and an amount of manual pressure being applied. For example, a personnel having a designation of D4 may refer to a specific staff member or physician such that for a manual pressure of less than 1 minute and a patient weighing less than 150 pounds has a risk score of 0.5, which may fall within a low risk range. In another example, a personnel having a designation of S2 may refer to a specific staff member or physician such that for a manual pressure of 2 minutes and a patient weighing approximately 150 pounds has a risk score of 1.5, which may fall within a medium risk range. In yet another example, a personnel having a designation of F1 may refer to a specific staff member or physician such that for a manual pressure of 6 minutes and a patient weighing approximately 300 pounds has a risk score of 12.4, which may fall within a high risk range. The risk scoring may be based on an ergonomic risk to the personnel. In some instances, a risk of low may comprise a limited risk of injury to personnel with repeated exposure. In some instances, a risk of medium may comprise a moderate risk of injury to personnel with repeated exposure. In some instances, a risk of high may comprise a significant risk of injury to personnel with repeated exposure.

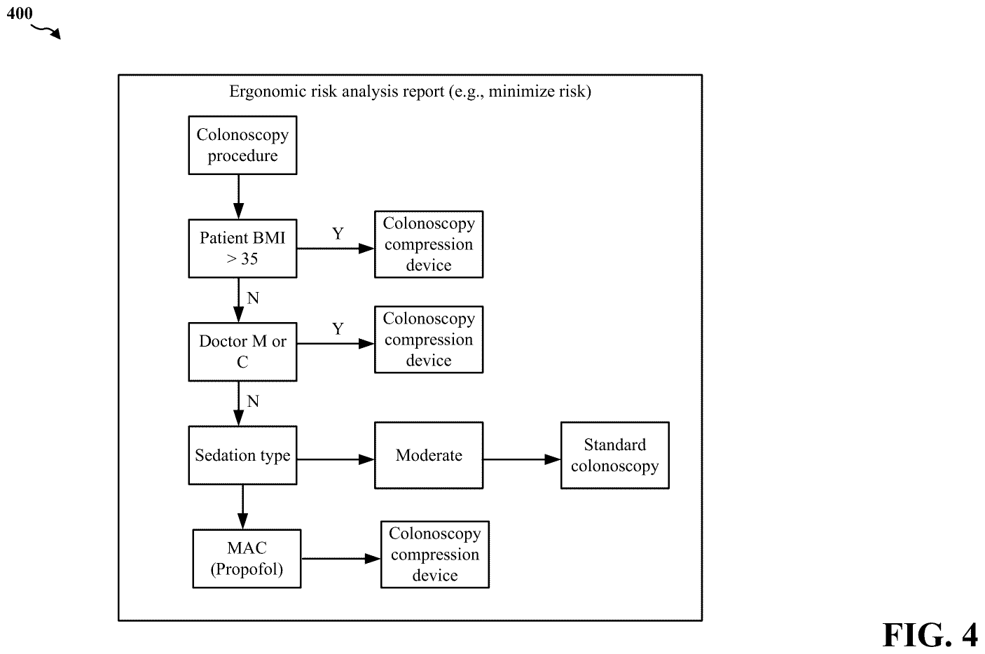

FIG. 4 illustrates a diagram 400 showing an example of an ergonomic risk analysis algorithm that be used for the risk analysis report. For example, the ergonomic risk analysis algorithm of FIG. 4 may provide an example of a flow chart that may determine ergonomic interventions based on the inputs. For example, for a colonoscopy procedure, the ergonomic risk analysis report may first determine whether the patient has a body mass index that exceeds a threshold (e.g., 35), if the patient has a body mass index that exceeds the threshold, the ergonomic risk analysis report may suggest that the patient utilize a colonoscopy compression device during the colonoscopy to minimize or mitigate the risk. However, if the patient has a body mass index that does not exceed the threshold, then the physician conducting the procedure may be reviewed. For example, if a particular physician is conducting the procedure and ergonomic interventions were previously utilized by patients when the particular physician performed procedures, or if previous procedures performed by the particular physician involved a medium or high risk, then the ergonomic risk analysis report may suggest that the patient utilize a colonoscopy compression device during the colonoscopy to minimize or mitigate the risk. However, if the particular physician is not involved in a specific procedure, then the sedation type utilized in the procedure may be reviewed. For example, if a sedation type is considered to be moderate, then the procedure may be considered to be a standard procedure. In another example, if the sedation type is MAC (e.g., propofol) then the ergonomic risk analysis report may suggest that the patient utilize a colonoscopy compression device during the colonoscopy to minimize or mitigate the risk. The flowchart of FIG. 4 is an example flowchart the reviews certain features of the patient, physician, and sedation type, and the disclosure is not intended to be limited to the aspects disclosed herein. In some aspects, the flowchart may review many other input features or factors of the patient, physician, staff, or the like that may be involved in the procedure to determine an ergonomic risk analysis report.

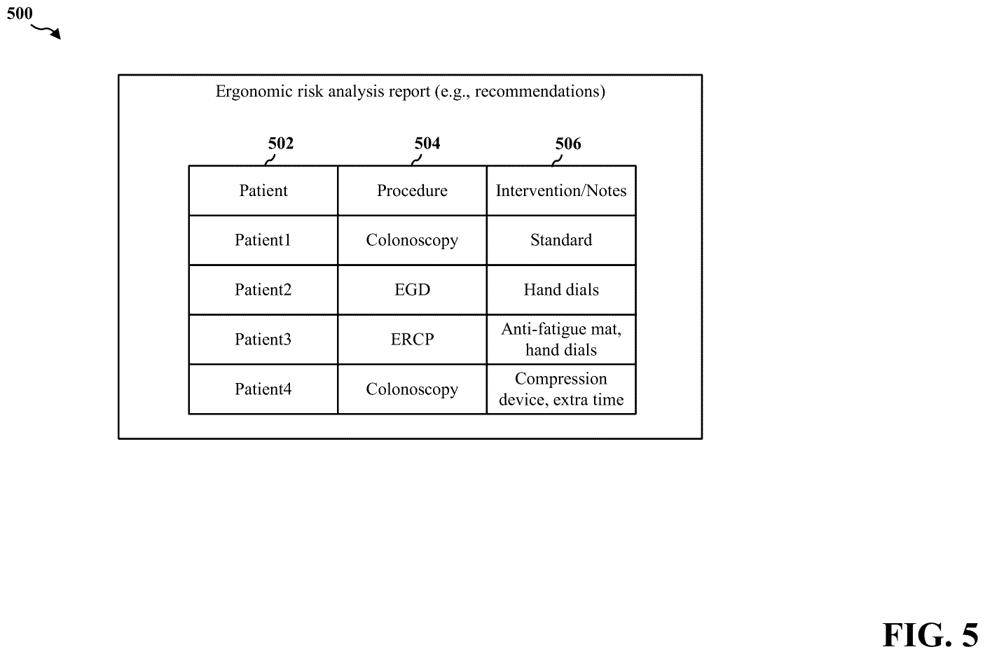

FIG. 5 illustrates a diagram 500 showing an example of an ergonomic risk analysis report (or alert) that can be provided by the system (e.g., 212 in FIG. 2 or 8). In some aspects, the ergonomic risk analysis report may comprise recommendations that are specific to each patient that is scheduled for a particular period of time. For example, the ergonomic risk analysis report may provide recommended intervention/notes 506 for each patient 502 that is scheduled to have a procedure 504 for a given day. For example, a first patient (e.g., patient1) may be scheduled for a colonoscopy as the first procedure for a given day. The colonoscopy for patient1 may be considered to be a standard procedure based on the intervention/notes 506. The second patient (e.g., patient2) may be scheduled for an esophagogastroduodenoscopy (EGD) shortly after patient 1, such that the recommended intervention/notes 506 may comprise hand dials that may reduce any risks in performing the EGD. The third patient (e.g., patient3) may be scheduled for an endoscopic retrograde cholangio pancreatography (ERCP) shortly after patient2, such that the recommended intervention/notes 506 may comprise anti-fatigue mat and/or hand dials that may reduce any risks in performing the ERCP. The fourth patient (e.g., patient4) may be scheduled for a colonoscopy shortly after patient3, such that the recommended intervention/notes 506 may comprise the patient utilize a compression device and/or allow for extra time that may reduce any risks in performing the colonoscopy. The physician and/or staff may be scheduled for multiple consecutive procedures and the ergonomic risk analysis report may provide recommendation that may mitigate or reduce any risks that may arise due in part to the scheduling of multiple consecutive procedures.

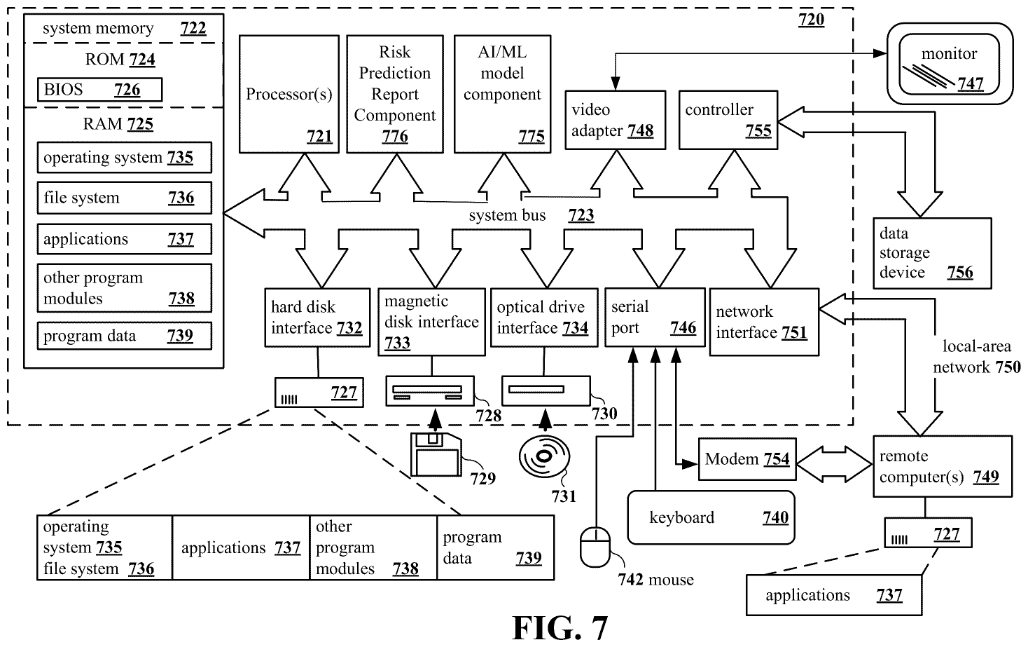

FIG. 6 is a flowchart 600 of a method of identifying risks in an endoscopic procedure. The method may be performed at a risk analysis system/medical alert system that may be configured to predict risks and generate alerts relating to endoscopic procedures. In some aspects, the method may be performed by an AI/ML model component 775, risk prediction report component 776, processor 721, and/or network interface 751 of a processing system, such as illustrated in FIG. 7. The method may include any of the aspects described in connection with FIGS. 1-5, for example. For example, the risk analysis system may correspond to risk analysis system 212.

The risk analysis system/medical alert system receives, at the communication interface of the medical alert system, a request for a risk prediction for a future endoscopic procedure, wherein the request includes procedural information related to at least one of historical endoscopic procedure information or proposed endoscopic procedural information for the future endoscopic procedure.

For example, as shown at 602, the risk analysis system/medical alert system may receive biographical information related to at least one of a patient, a physician, or staff involved in the endoscopic procedure. The risk analysis system may receive the biographical information at a communication interface of an AI model. In some aspects, the biographical information of the patient may comprise information related to physical features of the patient, medical conditions, or surgical history. In some aspects, the biographical information of the physician may comprise information related to physical conditions of the physician, medical conditions, or experience in performing endoscopic procedures. In some aspects, the biographical information of the staff may comprise information related to physical conditions of the physician, medical conditions, or experience in performing endoscopic procedures.

At 604, the risk analysis system/medical alert system may receive procedural information related to at least one of historical endoscopic procedure information or proposed endoscopic procedural information for the endoscopic procedure. The risk analysis system may receive the procedural information at the communication interface of the AI model. In some aspects, the procedural information may indicate information such as sedation method for the patient in previous procedures or in a scheduled procedure. In some aspects, the procedural information may indicate information related to equipment used (e.g., scope type) in the previous procedures or in the scheduled procedure.

At 606, the risk analysis system/medical alert system may receive preference information related to risk tolerances or goals of the endoscopic procedure. The requested risk prediction alert may then be based on the preference information. For example, the model may be calibrated, or a level of risk may be alerted or ignored, based on the received preference(s). The risk analysis system may receive the preference information at the communication interface of the AI model. In some aspects, identification of the one or more risks in performing the endoscopic procedure may be based at least on the preference information of the endoscopic procedure.

At 608, the risk analysis system/medical alert system may identify and output a recommendation of one or more ergonomic interventions. The recommendation may be reported in a report or may be flagged in real-time, e.g., via one or more of a light, a sound, a vibration, etc. The one or more ergonomic interventions may be implemented by at least one of the patient, the physician, or the other healthcare professional for the endoscopic procedure. The one or more ergonomic interventions implemented by at least one of the patient, the physician, or the staff for the endoscopic procedure may be based on identification of the one or more risks. In some aspects, the one or more ergonomic interventions may comprise at least one of the patient utilizing a compression device during the endoscopic procedure, receiving a sedation, or physical manipulation. In some aspects, the one or more ergonomic interventions may comprise at least one of the physician utilizing hand dials, an anti-fatigue mat, or increasing a scheduled time duration for the endoscopic procedure. In some aspects, the one or more ergonomic interventions may comprise at least one of the staff utilizing assistive equipment to assist in an application of external pressure on the patient or repositioning of the patient.

At 610, the risk analysis system/medical alert system may generate the risk prediction using a model, e.g., as described in connection with any of FIG. 2, 3, 4, 5, 7, or 8. In some aspects, the model may include an AI/ML model that is trained based on historical endoscopic procedure information, such as described in connection with FIG. 2. The model may generate the risk prediction based at least on the input received related to at least one of the patient, physician, or staff. The risk prediction may predict a level of risk to at least one of the physician or staff in performing the procedure.

In some aspects, the system may receive, via the communication interface, feedback including additional information based on the endoscopy procedure of the patient including one or more of outcome information for the patient, the physician, or the other medical professional. FIG. 2 illustrates an example of feedback 275 that is received by the system 212. In some aspects, the feedback may be received at (or from) a device at or near a treatment room for the endoscopy procedure via a user interface that displays a question about one or more of whether manual pressure was used, whether the patient was repositioned, or the effect on the physician or the other medical professional. Example aspects are described in connection with FIG. 9A and FIG. 9B. In some aspects, the feedback 275 may comprise video from a camera at a treatment room for the endoscopy procedure, and the model may be configured to associate a behavior, posture, or positioning identified from the video with a potential injury risk.

At 612, the risk analysis system/medical alert system may output a risk prediction alert associated with performance of the future endoscopic procedure. The risk prediction is based on risk information associated with one or more risks to at least one of the patient, the physician, or the other healthcare professional based at least on the biographical information or the proposed endoscopic procedural information.

FIG. 7 is a block diagram illustrating a general-purpose computer system 720 on which aspects of systems and methods for risk prediction and/or identification of interventions to mitigate risk using an AI/ML model, e.g., as described in connection with any of FIGS. 2-6 may be implemented in accordance with an example aspect. The computer system 720 can correspond to the physical server(s) on which an ergonomic risk analysis report is executed, for example, described herein. In some aspects, the system may include any of the aspects described in connection with FIG. 2. As described in connection with FIG. 2, in some aspects, the AI/ML model component 775 may be at a server that is accessed by one or more remote terminals to receive input for model training and/or model inference. The risk prediction report may be output via a communication interface from the server to one or more remote user terminals. In some aspects, the AI/ML inference may be performed at a remote server and provided to the user device. As an example, the prediction report may be accessed via a website or an application. In some aspects, the AI/ML model component may be local at a user's device or an application within a local network or user system. In some aspects, the AI/ML model component may be accessed as an add on program that adds additional functionality to a user system.

As shown, the computer system 720 (which may be a personal computer or a server) includes a central processing unit (e.g., 721), a system memory 722, and a system bus 723 connecting the various system components, including the memory associated with the central processing unit (e.g., 721). As will be appreciated by those of ordinary skill in the art, the system bus 723 may comprise a bus memory or bus memory controller, a peripheral bus, and a local bus that is able to interact with any other bus architecture. The system memory may include permanent memory (ROM) 724 and random-access memory (RAM) 725. The basic input/output system (BIOS) 726 may store the basic procedures for transfer of information between elements of the computer system 720, such as those at the time of loading the operating system with the use of the ROM 724.

The computer system 720 may also comprise a hard disk 727 for reading and writing data, a magnetic disk drive 728 for reading and writing on removable magnetic disks 729, and an optical drive 730 for reading and writing removable optical disks 731, such as CD-ROM, DVD-ROM and other optical media. The hard disk 727, the magnetic disk drive 728, and the optical drive 730 are connected to the system bus 723 across the hard disk interface 732, the magnetic disk interface 733, and the optical drive interface 734, respectively. The drives and the corresponding computer information media are power-independent modules for storage of computer instructions, data structures, program modules, and other data of the computer system 720.

An example aspect comprises a system that uses a hard disk 727, a removable magnetic disk 729 and a removable optical disk 731 connected to the system bus 723 via the controller 755. It will be understood by those of ordinary skill in the art that any type of media 756 that is able to store data in a form readable by a computer (solid state drives, flash memory cards, digital disks, random-access memory (RAM) and so on) may also be utilized.

The computer system 720 has a file system 736, in which the operating system 735 may be stored, as well as additional program applications 737, other program modules 738, and program data 739. A user of the computer system 720 may enter commands and information using keyboard 740, mouse 742, or any other input device known to those of ordinary skill in the art, such as, but not limited to, a microphone, joystick, game controller, scanner, etc. Such input devices typically plug into the computer system 720 through a serial port 746, which in turn is connected to the system bus, but those of ordinary skill in the art will appreciate that input devices may be also be connected in other ways, such as, without limitation, via a parallel port, a game port, or a universal serial bus (USB). A monitor 747 or other type of display device may also be connected to the system bus 723 across an interface, such as a video adapter 748. In addition to the monitor 747, the personal computer may be equipped with other peripheral output devices (not shown), such as loudspeakers, a printer, etc.

Computer system 720 may operate in a network environment, using a network connection to one or more remote computers 749. The remote computer (or computers) 749 may be local computer workstations or servers comprising most or all of the aforementioned elements in describing the nature of a computer system 720. Other devices may also be present in the computer network, such as, but not limited to, routers, network stations, peer devices or other network nodes.

Network connections can form a local-area computer network (LAN) 750 and a wide-area computer network (WAN). Such networks are used in corporate computer networks and internal company networks, and they generally have access to the Internet. In LAN or WAN networks, the computer system 720 is connected to the local-area network 750 across a network adapter or network interface 751. When networks are used, the computer system 720 may employ a modem 754 or other modules well known to those of ordinary skill in the art that enable communications with a wide-area computer network such as the Internet. The modem 754, which may be an internal or external device, may be connected to the system bus 723 by a serial port 746. It will be appreciated by those of ordinary skill in the art that said network connections are non-limiting examples of numerous well-understood ways of establishing a connection by one computer to another using communication modules.

In various aspects, the systems and methods described herein may be implemented in hardware, software, firmware, or any combination thereof. If implemented in software, the methods may be stored as one or more instructions or code on a non-transitory computer-readable medium. Computer-readable medium includes data storage. By way of example, and not limitation, such computer-readable medium can comprise RAM, ROM, EEPROM, CD-ROM, Flash memory or other types of electric, magnetic, or optical storage medium, or any other medium that can be used to carry or store desired program code in the form of instructions or data structures and that can be accessed by a processor of a general purpose computer.

In various aspects, the systems and methods described in the present disclosure can be addressed in terms of modules. The term “module” as used herein refers to a real-world device, component, or arrangement of components implemented using hardware, such as by an application specific integrated circuit (ASIC) or field-programmable gate array (FPGA), for example, or as a combination of hardware and software, such as by a microprocessor system and a set of instructions to implement the module's functionality, which (while being executed) transform the microprocessor system into a special-purpose device. A module, element, or component may also be implemented as a combination of the two, with particular functions facilitated by hardware alone, and other functions facilitated by a combination of hardware and software. In particular implementations, at least a portion, and in some cases, all, of a module, element, or component may be executed on one or more processors of a general purpose computer. Accordingly, each module may be realized in a variety of suitable configurations, and should not be limited to any particular implementation or example herein. An element, or any portion of an element, or any combination of elements may be implemented as a “processing system” that includes one or more processors. When multiple processors are implemented, the multiple processors may perform the functions individually or in combination. One or more processors in a processing system may execute stored instructions, which may be referred to as software, firmware, middleware, microcode, hardware description language, or otherwise, e.g., instructions, instruction sets, code, code segments, program code, programs, subprograms, software components, applications, software applications, software packages, routines, subroutines, objects, executables, threads of execution, procedures, functions, or any combination thereof. In one configuration, the AI/ML model component 775, risk prediction report component 776, and/or the computer system 720, and in particular, the file system 736 and/or the processor 721, is configured to perform the aspects of the flowchart in FIG. 6.

For example, the AI/ML model component may correspond to one or more components of the system 212 described in connection with FIG. 2 and/or FIG. 8. In some aspects, the AI/ML model may use machine-learning algorithms, deep-learning algorithms, neural networks, reinforcement learning, regression, boosting, or advanced signal processing methods for predicting risk and/or recommending interventions for a particular endoscopy procedure for a particular endoscopy patient.

Reinforcement learning is a type of machine learning that involves the concept of taking actions in an environment in order to maximize a reward. Reinforcement learning is a machine learning paradigm. Other paradigms include supervised learning and unsupervised learning. Basic reinforcement may be modeled as a Markov decision process (MDP) with a set of environment states and agent states, as well as a set of actions of the agent. A determination may be made about a likelihood of a state transition based on an action and a reward after the transition. The action selection by an agent may be modeled as a policy. The reinforcement learning may enable the agent to learn an optimal, or nearly-optimal, policy that maximizes a reward. Supervised learning may include learning a function that maps an input to an output based on example input-output pairs, which may be inferred from a set of training data, which may be referred to as training examples. The supervised learning algorithm analyzes the training data and provides an algorithm to map to new examples.

Regression analysis may include statistical analysis to estimate the relationships between a dependent variable (e.g., an outcome variable) and one or more independent variables. Linear regression is an example of a regression analysis. Non-linear regression models may also be used. Regression analysis may include estimating, or determining, relationships of cause between variables in a dataset. Boosting includes one or more algorithms for reducing variance or bias in supervised learning. Boosting may include iterative learning based on weak classifiers (e.g., that are somewhat correlated with a true classification) with respect to a distribution that is added to a strong classifier (e.g., that is more closely correlated with the true classification) in order to convert weak classifiers to stronger classifiers. The data weights may be readjusted through the process, e.g., related to accuracy.

Among others, examples of machine learning models or neural networks that may be included in the AI/ML model include, for example, artificial neural networks (ANN); decision tree learning; convolutional neural networks (CNNs); deep learning architectures in which an output of a first layer of neurons becomes an input to a second layer of neurons, and so forth; support vector machines (SVM), e.g., including a separating hyperplane (e.g., decision boundary) that categorizes data; regression analysis; Bayesian networks; genetic algorithms; deep convolutional networks (DCNs) configured with additional pooling and normalization layers; and deep belief networks (DBNs).

In some aspects, an example machine learning model, such as an artificial neural network (ANN), that includes an interconnected group of artificial neurons (e.g., neuron models) as nodes. Neuron model connections may be modeled as weights, in some aspects. A machine learning model may be adapted, e.g., based on external or internal information processed by the machine learning model. In some aspects, a machine learning model may include a non-linear statistical data model and/or a decision making model. Machine learning may model complex relationships between input data and output information.

A machine learning model may include multiple layers and/or operations that may be formed by concatenation of one or more of the referenced operations. Examples of operations that may be involved include extraction of various features of data, convolution operations, fully connected operations that may be activated or deactivated, compression, decompression, quantization, flattening, etc. The term layer may indicate an operation on input data. Weights, biases, coefficients, and operations may be adjusted in order to achieve an output closer to the target output. Weights and biases are examples of parameters of a trained machine learning model. Different layers of a machine learning model may be trained separately.

A variety of connectivity patterns, e.g., including any of feed-forward networks, hierarchical layers, recurrent architectures, feedback connections, etc., may be included in a machine learning model. Layer connections may be fully connected or locally connected. For a fully connected network, a first layer neuron may communicate an output to each neuron in a second layer. Each neuron in the second layer may receive input from each neuron in the first layer. For a locally connected network, a first layer neuron may be connected to a subset of neurons in the second layer, rather than to each neuron of the second layer. A convolutional network may be locally connected and may be configured with shared connection strengths associated with the inputs for each neuron in the second layer. In a locally connected layer of a network, each neuron in a layer may have the same, or a similar, connectivity pattern, yet having different connection strengths.

A machine learning model, artificial intelligence component, or neural network may be trained, such as training based on supervised learning. During training, the machine learning model may be presented with an input that the model uses to compute to produce an output. The actual output may be compared to a target output, and the difference may be used to adjust parameters (e.g., weights, biases, coefficients, etc.) of the machine learning model in order to provide an output closer to the target output. Before training, the output may not be correct or may be less accurate. A difference between the output and the target output, may be used to adjust weights of a machine learning model to align the output is more closely with the target.

A learning algorithm may calculate a gradient vector for adjustment of the weights. The gradient may indicate an amount by which the difference between the output and the target output would increase or decrease if the weight were adjusted. The weights, biases, or coefficients of the model may be adjusted until an achievable error rate stops decreasing or until the error rate has reached a target level.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents. In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

The teachings of each of U.S. application Ser. No. 18/924,923, entitled DEVICE FOR ENDOSCOPIC IMAGING FOR ENDOSCOPIC PROCEDURES, and filed on Oct. 23, 2024; U.S. application Ser. No. 18/799,990, entitled SHAPED ENDOSCOPY SUPPORT DEVICE INSERT, and filed on Aug. 9, 2024; U.S. application Ser. No. 18/799,999, entitled INSERT WITH VISCOUS FILLER FOR ENDOSCOPY SUPPORT, and filed on Aug. 9, 2024; U.S. application Ser. No. 17/180,676, entitled Endoscopy Band with Visual Indicator to Assist Placement, and filed on Feb. 19, 2021; U.S. application Ser. No. 16/818,877, entitled Endoscopy Band with Sigmoid Support Apparatus, and filed on Mar. 13, 2020; U.S. application Ser. No. 15/256,019, entitled “METHOD AND APPARATUS FOR ENHANCED VISUALIZATION DURING ENDOSCOPY,” and filed on Sep. 2, 2016; U.S. Provisional Application No. 62/214,747, entitled “IMPROVED BOWEL STABILITY AND ENHANCED VISUALIZATION DURING ENDOSCOPY” and filed on Sep. 4, 2015; and U.S. application Ser. No. 14/575,860, entitled “ENDOSCOPY BAND WITH SIGMOID SUPPORT APPARATUS,” and filed on Dec. 18, 2014; U.S. application Ser. No. 13/344,715, entitled “METHOD AND APPARATUS FOR TENSILE COLONOSCOPY COMPRESSION,” and filed on Jan. 6, 2012; U.S. Provisional Application Ser. No. 61/917,469, entitled “COLONOSCOPY BAND WITH SIGMOID SPLINT” and filed on Dec. 18, 2013; U.S. Provisional Application Ser. No. 61/944,658 entitled “ENDOSCOPY BAND WITH SIGMOID SUPPORT APPARATUS” and filed on Feb. 26, 2014; U.S. Provisional Application Ser. No. 62/978,797, entitled “Endoscopy Band With Visual Indicator to Assist Placement” and filed on Feb. 19, 2020, the contents of each of which are expressly incorporated by reference herein in their entirety.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of the skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

Example aspects of the present invention have now been described in accordance with the above advantages. It will be appreciated that these examples are merely illustrative of aspects of the present invention. Many variations and modifications will be apparent to those skilled in the art.

It is understood that the specific order or hierarchy of steps in the processes disclosed is an illustration of example approaches. Based upon design preferences, it is understood that the specific order or hierarchy of steps in the processes may be rearranged. Further, some steps may be combined or omitted. The accompanying method claims present elements of the various steps in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects.” Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

1. A medical alert system for predicted risks in endoscopic procedures, comprising:
a communication interface;
memory;
at least one processor coupled to the memory and configured to:
receive, at the communication interface of the medical alert system, a request for a risk prediction for a future endoscopic procedure, wherein the request includes procedural information related to at least one of historical endoscopic procedure information or proposed endoscopic procedural information for the future endoscopic procedure; and
output, via the communication interface, a risk prediction alert associated with performance of the future endoscopic procedure, wherein the risk prediction is based on risk information associated with one or more risks to at least one of a patient, a physician, or an additional healthcare professional based on at least one of biographical information or the proposed endoscopic procedural information.

2. The medical alert system of claim 1, wherein the at least one processor is further configured to:
generate the risk prediction using a model based on historical endoscopic procedural information.

3. The medical alert system of claim 2, wherein the model comprises an artificial intelligence (AI) or machine learning (ML) model that is trained based on the historical endoscopic procedural information.

4. The medical alert system of claim 3, wherein the at least one processor is configured to:
receive, via the communication interface, feedback information for an endoscopy procedure of the patient including one or more of outcome information for the patient, the physician, or the additional healthcare professional.

5. The medical alert system of claim 4, wherein the feedback information is received at a device at or near a treatment room for the endoscopy procedure via a user interface that displays a question about one or more of whether manual pressure was used, whether the patient was repositioned, or an effect on the physician or the additional healthcare professional.

6. The medical alert system of claim 4, wherein the feedback information comprises video from a camera at a treatment room for the endoscopy procedure, and wherein the model is configured to associate a behavior, posture, or positioning identified from the video with a potential injury risk.

7. The medical alert system of claim 1, wherein the at least one processor is further configured to:
receive, via the communication interface, preference information related to risk tolerances or goals of an endoscopy procedure, wherein the risk prediction alert is based on the preference information received at the communication interface.

8. The medical alert system of claim 1, wherein the at least one processor is further configured to:
output a recommendation of one or more ergonomic interventions to be implemented by at least one of the patient, the physician, or the additional healthcare professional for an endoscopic procedure based on identification of the one or more risks.

9. The medical alert system of claim 8, wherein the one or more ergonomic interventions comprises at least one of the patient utilizing a compression device during the endoscopic procedure, receiving a sedation, or physical manipulation.

10. The medical alert system of claim 8, wherein the one or more ergonomic interventions comprises at least one of the physician utilizing hand dials, an anti-fatigue mat, or increasing a scheduled time duration for the endoscopic procedure.

11. The medical alert system of claim 8, wherein the one or more ergonomic interventions comprises at least one of the additional healthcare professional utilizing assistive equipment to assist in an application of external pressure on the patient or repositioning of the patient.

12. The medical alert system of claim 1, wherein the biographical information of the patient comprises information related to one or more of physical features of the patient, medical conditions of the patient, or surgical history of the patient.

13. The medical alert system of claim 1, wherein the biographical information of the physician comprises information related to one or more of physical conditions of the physician, medical conditions of the physician, or experience of the physician in performing multiple endoscopic procedures.

14. The medical alert system of claim 1, wherein the biographical information of the additional healthcare professional comprises information related to physical conditions of the additional healthcare professional, medical conditions of the additional healthcare professional, or experience of the additional healthcare professional in performing multiple endoscopic procedures.

15. The medical alert system of claim 1, further comprising:
receiving, via the communication interface of the medical alert system, the biographical information related to at least one of the patient, the physician, or the additional healthcare professional involved in an endoscopic procedure.

16. A method for providing a medical alert via a medical alert system for endoscopic procedures, comprising:
receiving, at a communication interface of the medical alert system, proposed endoscopic procedural information for a future endoscopic procedure; and
outputting, via the communication interface, a risk prediction alert associated with performance of the future endoscopic procedure, wherein the risk prediction alert is based on risk information associated with one or more risks to at least one of a patient, a physician, or an additional healthcare professional based on at least one of biographical information or the proposed endoscopic procedural information.

17. A non-transitory computer-readable medium storing code for a medical alert system for endoscopic procedures, the code when executed by one or more processors causes the one or more processors to:
receive, at a communication interface of the medical alert system, proposed endoscopic procedural information for a future endoscopic procedure; and
output, via the communication interface, a risk prediction alert associated with performance of the future endoscopic procedure, wherein the risk prediction alert is based on risk information associated with one or more risks to at least one of a patient, a physician, or an additional healthcare professional based on at least one of biographical information or the proposed endoscopic procedural information.
